(19) United States
(12) Patent Application Publication
(10) Pub. No.: US 2009/0129991 A1
(43) Pub. Date: May 21, 2009

(54) TOY HAVING AIR PURIFICATION FEATURES AND METHOD FOR MAKING SAME

(76) Inventor: Jasjit S. Dhillon, Oak Brook, IL (US)

Correspondence Address: MICHAEL P. MAZZA, LLC, 686 CRESCENT BLVD., GLEN ELYN, IL, 60137, US

(21) Appl. No.: 12/358,333

(22) Filed: Jan. 23, 2009

Related U.S. Application Data
(63) Continuation-in-part of application No. 11/162,547, filed on Sep. 14, 2005.
Application No. 11/162,547 is a continuation-in-part of application No. 10/940,145, filed on Sep. 14, 2004, now abandoned.

(51) Int. Cl. A61L 2/08; A63H 3/02; B23P 19/04

(52) U.S. Cl. 422/105; 446/369; 29/700; 422/121

(57) ABSTRACT

A toy such as a plush toy having various sanitizing features is disclosed, The toy of the present invention optionally includes an air purifying device such as an ionizing device or an ozone device disposed within an interior cavity of the toy. The toy optionally includes a visco-elastic material. The visco-elastic material may be infused with an anti-microbial nano-material such as a silver nano-material. Furthermore, an outer material layer of the toy optionally includes an antimicrobial agent, and can be removable and washable to reduce or eliminate air particulate. An optional liner may be hypoallergenic. The air purifying device is optionally activated via a sensor, such as a motion or heat sensor, and is optionally deactivated via a timer.

### CROSS-REFERENCE TO RELATED APPLICATIONS

This is a continuation-in-part of application Ser. No. 11/162,547 filed on Sep. 14, 2005, which is a continuation-in-part of U.S. Ser. No. 10/940,145 filed on Sep. 14, 2004 which is hereby incorporated by reference in its entirety.

### FIELD OF THE INVENTION

The present invention relates to plush toys. Specifically, the present invention relates to a plush toy having ionizing or other sanitizing properties and optionally has shape retaining properties.

### DESCRIPTION OF THE RELATED ART

Plush toy is a generic term generally referring to stuffed animals or similar stuffed toys having a fabric covering filled or stuffed with a filling material. Typically the fabric covering is soft, furry, or fluffy such that the toy feels pleasant to someone holding the toy. The filling for plush toys can be any suitable material, but is often foam or fabric based.

Plush toys often become a breeding ground for bacteria, germs, dust mites, and other potentially harmful agents. It is not uncommon for children, the most frequent users of plush toys, to have the toy in their mouth one moment, drag the toy across the floor the next moment, and shortly thereafter sleep or cuddle up with the toy. Despite a caregiver's best efforts; it is too often a losing battle to try to keep the plush toys clean.

Some attempts have been made to create a plush toy that fights the spread of germs. For example, see U.S. Pat. No. 6,240,879, issued to Denesuk et al., which discloses a plush toy that has a microbe-inhibiting agent applied to the outer casing and or the filling.

In addition to the application of antimicrobial agents, the use of ionizers or other sanitizers is relevant to the present invention. In ionizing, contaminants are drawn into an apparatus by the movement of air around the apparatus and through the use of an electric field an electric charge, either positive or negative, is imparted to the contaminants. The contaminants can include dust, smoke, vapors, bacteria and the like. The charged contaminants are then attracted to plates or similar devices within the apparatus that have an opposite charge. Thus the contaminants are removed from the air and trapped in the ionizing device. This technology is most commonly applied in commercially available electrostatic air cleaners. However, this technology has not been applied to plush toys.

While ionizing is new to plush toys, there are devices that use plush toys or pillows to deliver medicine or other agents to a user in close proximity to the toy or pillow. For example, U.S. Pat. No. 6,688,305, issued to Perry, discloses a therapeutic aid which has a body (shown shaped like a plush toy) having a perimeter wall defining an inner space. A treatment member is insertable into the inner space of the body member. The treatment member is designed to emit therapeutic vapors through the body such that the vapors ease the cold symptoms of a child when the body is placed in proximity to the child. Similarly, U.S. Pat. No. 618,210, issued to Shakespeare, discloses a holder for medicine cups, where the holder is attached to a pillow and the medicine cup is secured in the holder. Vapors from the medicine in the medicine cup are allowed to escape the cup and holder and reach a person using the pillow. U.S. Pat. No. 290,608, issued to Snyder, also discloses a medicinal inhaler that attaches to a pillow.

Another technology applicable to plush toys is the use of shape retaining or resilient materials in the filling or other parts of the toy. The use of these materials can increase enjoyment of the toy over time as the material helps the toy retain its original shape. There are toys that have shape retaining properties. For example, U.S. Pat. No. 5,026,054, issued to fisher, et al., discloses a toy ball having a highly plasticized polymeric core which is resiliently deformable due to its highly plasticized state. U.S. Pat. No. 2,830,402 discloses an ornamental toy having retarded resiliency, in which the toy slowly returns to its original shape. U.S. Pat. No. 4,170,086, issued to Hills, discloses a stuffed animal toy having a porous, flexible and resilient material included in the outer covering. The resilient material stiffens and imparts a planar memory to the outer covering. Additionally, U.S. Pat. No. 4,505,687, issued to Munro, discloses a form retaining stuffed figurine, wherein an elastic member is extending along part of the outer covering of the figurine. The elastic member allows the figurine to be manually deformed and then return to a predetermined shape when the deforming forces are removed.

One type of resilient material heretofore unused in plush toys is visco-elastic foam. Visco-elastic foam is material originally developed by NASA which is temperature sensitive and deforms under pressure. When the pressure is removed it slowly returns to its original shape. At present, visco-elastic foam is found in various brands of mattresses, mattress covers, and pillows.

There remains a need for a plush toy that combines the germ and contaminant fighting capabilities of antimicrobial treatments with an ionizer or similar sanitizer. Furthermore there remains a need for a plush toy that includes the shape retaining properties of visco-elastic foam. Thus, it would be advantageous to provide a plush toy that combines the germ and contaminant fighting capabilities of antimicrobial treatments with an ionizer and optionally has shape retaining properties.

### SUMMARY OF THE INVENTION

In view of the insufficiencies discussed above, it is an object of the present invention to provide a plush toy or children's toy having various features and advantages.

The present invention is a plush toy or children's toy having various features and advantages. In one embodiment the invention is a plush toy having an air purifying device disposed therein. The air purifying device is optionally an ionizing device, ozone air purification device, or other air purification device. Optionally, the device is removable, such that a power supply such as batteries can be replaced or recharged.

The device of the present invention is optionally formed of visco-elastic foam, such that it is deformable under pressure, yet returns to its original shape when the pressure is removed. Pathways such as holes, tubes, or natural material pores and openings allow air from the surface of the toy to enter and become purified via the device.

Furthermore, the device of the present invention optionally includes an outer material layer incorporating an antimicrobial agent. The outer material layer is optionally removable, such as via a zipper opening, hook and loop fastener opening, or other suitable means. An inner lining may also be provided. The inner lining or the outer material layer may be hypoallergenic. The foam filling may also be infused with an anti-microbial agent. One type of antimicrobial agent which can be used is a nanotechnology material such as a silver or copper nano-material.

In one embodiment of the invention the air purifying device can be activated by a sensor, such as a heat or motion sensor. Thus, the air purifying device can be selectively activated while a child is playing with the toy. Furthermore, the air purifying device is optionally deactivated via a timer. For example, the timer can deactivate the device after a fixed amount of time of no input from the sensor.

The toy of the present invention can range from a stuffed animal type toy, a pillow, a plastic toy figure, or any other suitable toy. Among the numerous advantages of the various embodiments of the present invention, the toy provides a sanitary and continually sanitizing environment which can reduce or eliminate germs on and within the toy, as well as the surrounding environment. Such features are particularly useful for reducing illness and reducing the risks of contamination from a shared toy.

Other features and advantages of the invention will be apparent from the following detailed description take in conjunction with the following drawings, wherein like reference numerals represent like features.

### BRIEF DESCRIPTION OF THE DRAWINGS

FIG. 1 is a side view of a plush toy of the present invention showing pathways from the surface to the interior cavity.

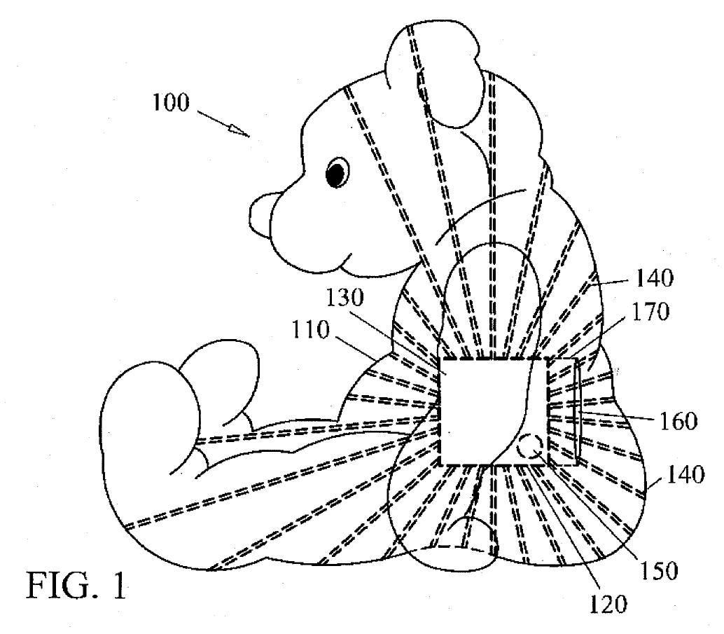

FIG. 2 is a side view of the present invention showing the features shown in FIG. 1, but showing the plush toy in a deformed condition.

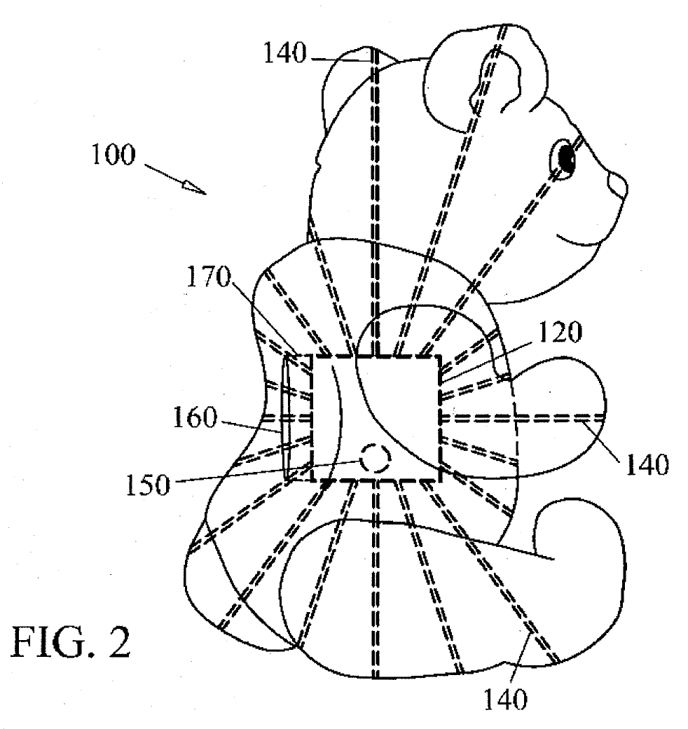

FIG. 3 is a side view of another embodiment of the present invention without any deformation.

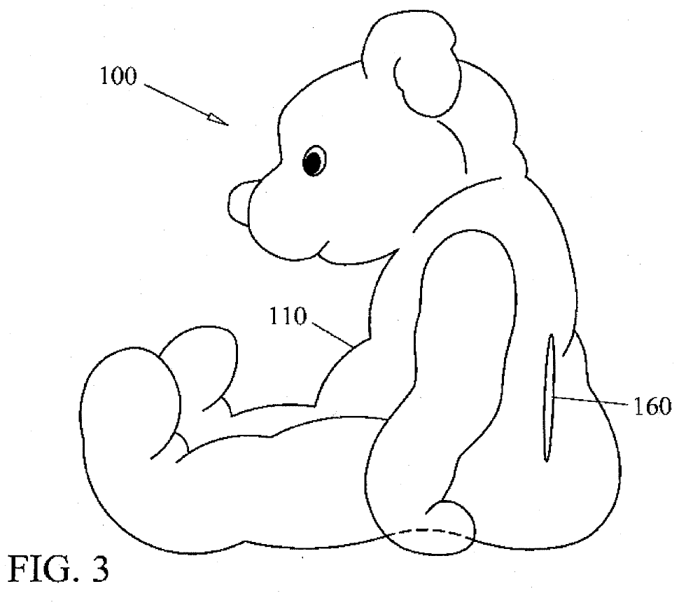

FIG. 4 is a side view of another embodiment of the present invention showing the plush toy in deformed condition.

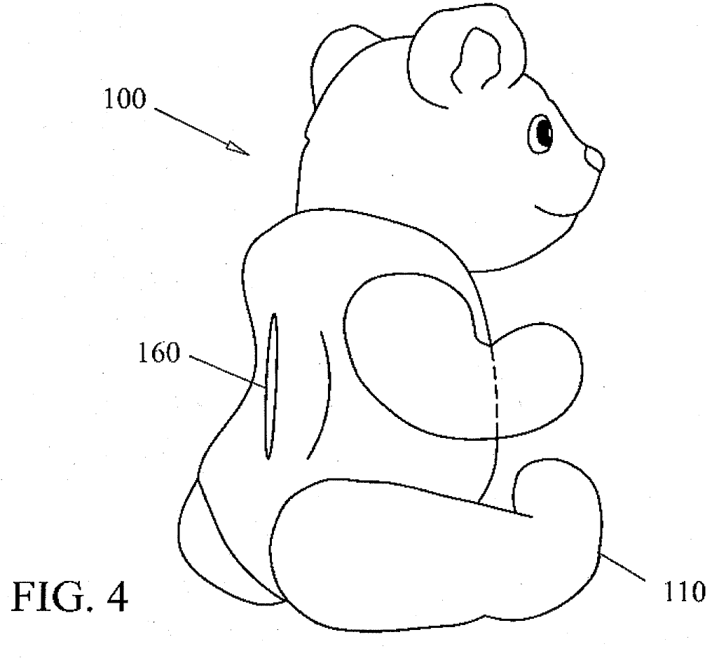

FIG. 5 is a side view of another embodiment of the present invention without any deformation, showing an alternative location for the air purifying device.

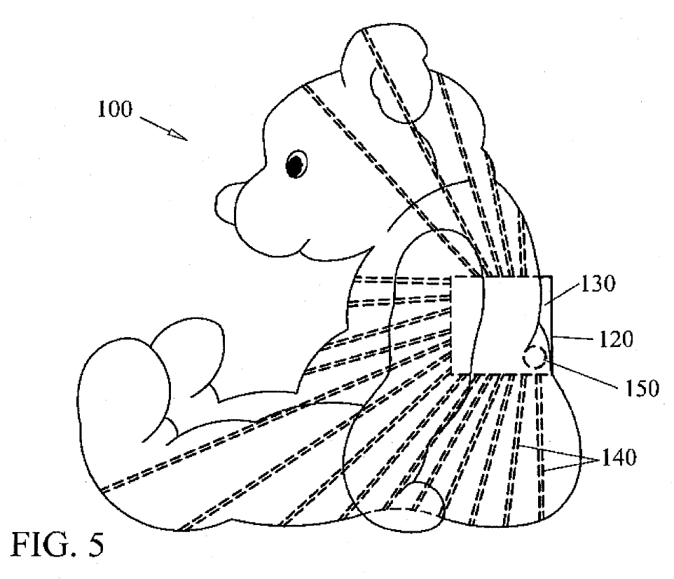

### DETAILED DESCRIPTION OF THE INVENTION

While this invention is susceptible of embodiments in many different forms, there are shown in the drawings, and will herein be described in detail, preferred embodiments of the invention with the understanding that the present disclosure is to be considered as an exemplification of the principles of the invention and is not intended to limit the broad aspect of the invention to the embodiments illustrated.

A toy 100, such as a plush toy, a pillow, a plastic toy, or any other suitable toy is disclosed, having a surface 110 forming the shape of a figure. In various embodiments, the toy 100 comprises an interior cavity 120. An air purifying device 130 is disposed within the interior cavity 120. The air purifying device 130 can be located anywhere within the toy 100. Alternatively, the air purifying device 130 could be located adjacent the surface 110, or even exterior thereto.

A plurality of pathways 140 extend from the interior cavity 120 to the surface 110. The passageways 140 are either natural passageways 140 which are formed by the natural openings or pores within the materials of the toy 100, or formed passageways 140 such as formed holes or tubules. The passageways 140 allow air to travel from the surface 110 or the surrounding environment to the air purifying device 130. Any other means of allowing access between the surface 110 and the device 130 to enable the device 130 to be effective in purifying air at the surface of the toy 100 is also contemplated within the scope of the present invention.

The purifying device 130 can be any suitable air purifying device which removes germs or contaminants, For example, the air purifying device 130 is optionally an ionizing device 130 or an ozone device 130 however, any other suitable device 130 is also contemplated.

The air purifying device 130 is optionally activated via a sensor 150, such as a motion sensor 150 or heat sensor 150, Thus, the device may be activated in the presence of a child or upon usage. Furthermore, the sensor 150 is optionally deactivated via a timer after a fixed amount of time has lapsed without input from sensor 150.

In various embodiments, the toy 100 includes an antimicrobial agent incorporated into the surface 110 or the filling. This provides for further germ elimination. The surface material 110, or an inner lining, may also be hypoallergenic. The surface 110 may comprise an outer material layer 110 which is removable and washable. The outer material layer 110 may be removable via an opening 160 such as a zipper opening, a hook and loop fastener opening, or any other suitable means. Any suitable power source is contemplated within the scope of the invention to power the air purifying device. The air purifying device 130 may also be removable to replace or recharge batteries or other power supplies. Alternatively, a passageway 170 may be provided to access the device 130 and the power supply for replacing or recharging, without removing the device 130 or even the power supply. Conceivably, the power supply might even be recharged via induction without the need for direct access to the power supply.

While the toy 100 might be filled with any suitable material, in certain embodiments, the toy 100 comprises a visco-elastic foam filling 150. Optionally, the invention may comprise the visco-elastic foam filled toy 100 in combination with an outer surface material 110 or inner lining incorporating an antimicrobial agent or a hypoallergenic material. Again, the outer material layer 110 may be removable and washable.

The anti-microbial agent of the present invention may be any suitable anti-microbial agent. However, in various preferred embodiments, the anti-microbial agent is a material produced using nano-technology, also referred to herein as “nano-material.” Such an agent is produced by using very tiny particles of a material having anti-microbial properties. In various preferred embodiments, a nano-material comprising silver nano-particles or copper nano-particles is used. These particles can be used to create a product which is anti-mold, anti-mite, and anti-bacterial, Such a material can be infused into the foam of the toy.

A method for manufacturing the plush toy is also disclosed. First, the anti-microbial agent (AMA) may be selected, such as a silver ion known as Sil Pure available from Ultra Fresh Thomas Research and Associates. A visco-elastic foam (VEF) may then by selected, but such as any of a variety of VEFs from China. The AMA may then be mixed with the VEF before the VEF is heated and foamed. The VEF with the dispersed AMA may now be poured into a mold forming a desired shape of the plush toy. The VEF may be heated for a suitable time period to form the foam. Once this interior portion of the toy has been formed, an outer cover of microfiber is selected and sewn to fit the visco-elastic foam mold shape. A suitable microfiber material, such as Micro Plush from China may be selected. In another preferred embodiment, the AMA may be a nanotechnology agent. In yet another preferred embodiments of manufacturing the plush toy, the AMA may be a metal nano-material. In yet another preferred embodiment, the microfiber may be an AMA or hypoallergenic.

While the specific embodiments have been illustrated and described, numerous modifications are possible without significantly departing from the spirit of the invention, and the scope of protection is only limited by the scope of the accompanying claims.

What is claimed is:

1. A plush toy comprising:
a surface forming the shape of a figure,
an interior cavity,
an antimicrobial agent incorporated into said surface, and
wherein at least a portion of said plush toy is formed of a visco-elastic foam.

2. The plush toy according to claim 1, further comprising an air purifying device disposed with said interior cavity, wherein said purifying device is an air ionizing device.

3. The plush toy according to claim 2, wherein said purifying device is an ozone purification device.

4. The plush toy according to claim 2, wherein said air purifying device is activated via a sensor.

5. The plush toy according to claim 4, wherein said sensor is a motion sensor.

6. The plush toy according to claim 4, wherein said sensor is a heat sensor.

7. The plush toy according to claim 4, wherein said air purifying device is deactivated via a timer, wherein said timer deactivates said air purifying device after a fixed amount of time of no input from said sensor.

8. The plush toy according to claim 7, wherein said toy is hypoallergenic.

9. The plush toy according to claim 7, wherein said visco-elastic foam is infused with an anti-microbial agent.

10. The plush toy according to claim 9, wherein said agent is a metal nano-material.

11. The plush toy according to claim 10, wherein said toy is hypoallergenic.

12. The plush toy according to claim 1, wherein said surface comprises a layer of material which is removable and washable.

13. The plush toy according to claim 12, wherein said layer of material comprises a zipper opening.

14. A plush toy comprising:
an outer material layer forming the shape of a figure, and a visco-elastic filling which is formed of a single piece and is substantially in the shape of the outer material layer, and which is deformable and can return to its original shape, wherein said filling is infused with an antimicrobial agent.

15. The plush toy according to claim 14, wherein said antimicrobial agent is a nanotechnology agent.

16. The plush toy according to claim 14, wherein said antimicrobial agent is a metal nano-material.

17. The plush toy according to claim 16, wherein said metal nano-material is a silver nano-material.

18. The plush toy according to claim 14, where said outer material layer is removable and washable.

19. The plush toy according to claim 14, wherein said outer material layer comprises an antimicrobial agent.

20. The plush toy according to claim 14, wherein said outer material layer is hypoallergenic.

21. The plush toy according to claim 14, further comprising a liner encasing said filling, wherein said liner is hypoallergenic.

22. The plush toy according to claim 1, further comprising a plurality of pathways extending from said interior cavity to said surface, wherein said pathways comprise substantially continuous and substantially straight holes.

23. The plush toy according to claim 22, wherein said pathways comprise natural openings within filling material.

24. The plush toy according to claim 2, wherein said air purifying device is removable.

25. The plush toy according to claim 2, further comprising an access passage from said surface to said air purifying device to enable maintenance of a power source.

26. The method of manufacturing a plush toy, comprising:
selecting an antimicrobial agent;
selecting a visco-elastic foam;
mixing the antimicrobial agent with the visco-elastic foam before the visco-elastic foam is heated and foamed;
pouring the visco-elastic foam dispersed with the antimicrobial agent into the mold forming the desired shape of the plush toy; and
heating the visco-elastic foam dispersed with the antimicrobial agent.

27. The method of claim 26, further comprising the steps of:
selecting a microfiber; and
sewing the microfiber to fit the visco-elastic foam dispersed with the antimicrobial agent shaped from the mold.

28. The method of claim 27 wherein the antimicrobial agent comprises a nanotechnology agent.

29. The method of claim 27 wherein an antimicrobial agent that is a metal nano-material.

30. The method of claim 27 wherein the microfiber comprises an antimicrobial agent.

31. The method of claim 27 wherein the microfiber is hypoallergenic.
